(19) United States
(12) Patent Application Publication
(10) Pub. No.: US 2011/0028865 A1
(43) Pub. Date: Feb. 3, 2011

(54) Inertial Sensor Kinematic Coupling

(75) Inventors: Hendrik Johannes Luinge, Enschede (NL); Daniel Roetenberg, Enschede (NL); Per Johan Slycke, Schalkhaar (NL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Xsens Technologies, B.V., Enschede (NL)

(21) Appl. No.: 12/534,526

(22) Filed: Aug. 3, 2009

(51) Int. Cl. A61B 5/11

(52) U.S. Cl. 600/595

(57) ABSTRACT

A method is disclosed for measuring the motion of an object, composed of multiple segments connected by joints, via the estimation of the 3D orientation of the object segments relative to one another without dependence on a magnetic field as a reference for heading. The method includes first applying a plurality of inertial sensor units to the segments of the object, e.g., a user thigh, shank, foot, etc. Next an approximation of the distance between each inertial sensor unit and at least one adjacent joint is provided and the joint is subjected to an acceleration, e.g., as the user takes a step or two. The relative orientations of the segments are calculated and the orientations are used to form an estimation of the 3D orientation of the object segments relative to one another without using the local magnetic field as a reference for heading.

### FIELD OF THE INVENTION

The invention relates to a motion tracking system for tracking an object composed of object parts, connected by joints, in a three-dimensional space, and in particular, to a motion tracking system for tracking the movements of a human body.

### BACKGROUND OF THE INVENTION

Measurement of motion with a high resolution is important for many medical, sports and ergonomic applications. Further, in the film and computer game market, there is a great need for motion data for the purpose of advanced animation and special effects. Additionally, motion data is also important in Virtual Reality (VR) and Augmented Reality (AR) applications for training and simulation. Finally, real-time 3D motion data is of great importance for control and stabilization of robots and robotic devices.

There are a number of technologies available for tracking and recording 3D motion data. They generally require that an infrastructure be constructed around the object to be tracked. For example, one such system is an optical system that uses a large number of cameras, fixedly arranged around the object for which the motion is to be tracked. However, such optical measuring systems can only track the motion of an object in the volume which is recorded with the cameras. Moreover, a camera system suffers from occlusion when the view of the camera of the object is obstructed by another object, or when one or more cameras perform poorly, e.g., due to light conditions.

Systems which track position and orientation on the basis of generating magnetic fields and detecting the generated field with a magnetometer also require an extensive infrastructure around the object of interest. While such magnetic systems do not suffer from occlusion and will work in any light condition, they are nonetheless relatively sensitive to magnetic disturbances. Further, these systems need relatively large transmitters due to the rapid decrease in magnetic field strength over distance.

Other systems rely on mechanical or optical goniometers to estimate joint angles. However, such systems lack the capability to provide an orientation with respect to an external reference system, e.g., earth. Moreover, the mechanical coupling to the body of interest is cumbersome. While systems based on ultra-sonic sensors do not share all of the above problems, they are prone to disturbances such as temperature and humidity of the air as well as wind and other ultra-sonic sources. In addition, the range of such systems is often relatively limited and thus the amount of installed infrastructure is demanding.

In many cases, it is desired to measure motion data of body segments in an ambulatory manner, i.e., in any place, on short notice, without extensively preparing the environment. A technology which is suitable for this makes use of inertial sensors in combination with earth magnetic field sensors. Inertial sensors, such as gyroscopes and accelerometers, measure their own motion independently of other systems. An external force such as the measured gravitational acceleration can be used to provide a reference direction. In particular, the magnetic field sensors determine the earth's magnetic field as a reference for the forward direction in the horizontal plane (north), also known as “heading.”

The sensors measure the motion of the segment on which they are attached, independently of other system with respect to an earth-fixed reference system. The sensors consist of gyroscopes, which measure angular velocities, accelerometers, which measure accelerations including gravity, and magnetometers measuring the earth magnetic field. When it is known to which body segment a sensor is attached, and when the orientation of the sensor with respect to the segments and joints is known, the orientation of the segments can be expressed in the global frame. By using the calculated orientations of individual body segments and the knowledge about the segment lengths, orientation between segments can be estimated and a position of the segments can be derived under strict assumptions of a linked kinematic chain (constrained articulated model). This method is well-known in the art and assumes a fully constrained articulated rigid body in which the joints only have rotational degrees of freedom.

The need to utilize the earth magnetic field as a reference is cumbersome, since the earth magnetic field can be heavily distorted inside buildings, or in the vicinity of cars, bikes, furniture and other objects containing magnetic materials or generating their own magnetic fields, such as motors, loudspeakers, TVs, etc.

Additionally, it is necessary to know the length of the rigid bodies connecting the joints with accuracy in order to accurately compute the motion of a constrained articulated rigid body. However, it is often impossible to accurately measure the distance between the joints since the internal point of rotation for each joint is not exposed and easily accessible. For example, the rotation joint inside the human knee cannot easily be measured from the outside. An additional complication for externally measuring the location of a joint is that the joint location may not be fixed over time, but may change depending upon the motion being executed. This is the case with respect to the human knee and shoulder for example. Methods of calibrating such a kinematic chain to accurately calibrate the relative positions of the joints are known in the art, however, such methods still rely on accurate orientation sensing, which is cumbersome in areas with distorted Earth magnetic field as described above, when utilizing inertial and magnetic sensing units.

### BRIEF SUMMARY OF THE INVENTION

It is an object of the invention to provide a system, in which positions and orientations of an object composed of parts linked by joints, and in particular the positions and orientations of the object parts relative to one another, can be measured with respect to each other in any place in an ambulatory manner, without dependence on the Earth magnetic field as a reference for rotation around the vertical (heading).

It is a further object of the invention to provide a system in which the distance between the joints linking the object parts can be estimated accurately while using the system, or as part of a separate calibration procedure.

Other objects and features of the invention will be appreciated from reading the following description in conjunction with the included drawings of which:

### BRIEF DESCRIPTION OF THE SEVERAL VIEWS OF THE DRAWINGS

FIG. 1 is a schematic cross-sectional diagram of a multi-segment jointed body with respect to which embodiments of the invention may be applied;

FIG. 2 is a photographic view of a test bed device within which an embodiment of the invention was implemented for test purposes;

FIG. 3 is a collection of data plots showing calibrated data of a sensor A within the device of FIG. 2 in accordance with an embodiment of the invention;

FIG. 4 is a collection of data plots showing the relative orientation (the orientation of sensor B with respect to sensor A) during testing, expressed in sensor A frame and expressed in the Global frame in accordance with an embodiment of the invention;

FIG. 5 is a collection of data plots showing measurement data of sensor A for a test wherein the prosthesis test bed of FIG. 2 was rotated around the hinge, around sensor A and around the shoulder with the prosthesis held in extension of the arm, and data gathered and processed in accordance with an embodiment of the invention;

FIG. 6 is a collection of data plots showing the relative heading estimation, expressed in Sensor A frame and expressed in Global frame, for the three different rotations of FIG. 5 in accordance with an embodiment of the invention;

FIG. 7 is a collection of data plots showing calibrated data of sensor A for a test wherein the prosthesis was translated along the x-, y- and z-axes, the gathering and processing of data being in accordance with an embodiment of the invention;

FIG. 8 is a collection of data plots showing relative heading estimation for several movements of the prosthesis in accordance with an embodiment of the invention;

FIG. 9 is a schematic diagram showing a leg with a knee joint connecting the upper leg (thigh) with the lower leg (shank) and an ankle joint connecting the shank with the foot;

FIG. 10 is a schematic showing the processing flow of the algorithm, including the use of a set of predetermined parameters for the algorithm according to an embodiment of the invention;

FIG. 11 is a a schematic diagram showing the relations between the ankle joint, sensor B (attached to the shank) and sensor C (attached to the foot) in keeping with the model of FIG. 9; and

FIG. 12 is a schematic diagram showing a model of two rigid segments A and B connected by a joint, with respect to which the disclosed principles may be applied.

### DETAILED DESCRIPTION OF THE INVENTION

The kinematic coupling (KiC) algorithm calculates (relative) orientation of two segments on each side of a joint. An inertial measurement unit aka IMU (3D accelerometer, 3D gyroscope, optionally equipped with a 3D magnetometer) is rigidly attached to each body segment. Only limited a-priori knowledge about the joint connection is needed to accurately determine the joint angle. This relative orientation between the two segments is essentially determined without using the local magnetic field as a reference for heading but using information derived from the joint acceleration.

The following initial assumptions are made:
rA en rB, the joint expressed in the sensor frame A and B, respectively, are fixed.
The Global frame is defined by X pointing to the north, Y pointing to the west and Z pointing up.
The acceleration and angular velocity of segment A and segment B are measured by the sensors attached to these segments.
The initial sensor orientations are calculated with use of the measured init acceleration and measured init magnetic field, or alternatively, using an arbitrary initial estimate.
The acceleration due to gravity is assumed known and constant. Furthermore, in the derived equations below, no account is taken for the Earth angular velocity.

The state vector is defined by:

xₜ=[^{G}Δpₜ ^{G}Δvₜ ^{G}a_{A,lowpass,t} ^{S}^{_{A}}θ_{ε,A,t} ^{S}^{_{A}}b_{A,t} ^{S}^{_{B}}θ_{ε,B,t} ^{S}^{_{B}}b_{B,t}]
^{G}Δpₜ=The relative position expressed in global frame
^{G}Δvₜ=The relative velocity expressed in global frame With
^{G}a_{A,lowpass,t}=The low pass acceleration of sensor A expressed in global frame
^{S}^{_{A}}θ_{ε,A,t}=The orientation error of sensor A expressed in ‘sensor A’ frame
^{S}^{_{A}}b_{ε,A,t}=The gyroscope bias of sensor A expressed in ‘sensor A’ frame
^{S}^{_{B}}θ_{ε,B,t}=The orientation error of sensor B expressed in ‘sensor B’ frame
^{S}^{_{B}}b_{,t}=The gyroscope bias of sensor B expressed in ‘sensor B’ frame
   Correct the angular velocity with the estimated gyroscope offset.

^{S}^{_{A}}ω_{A,t}=^{S}^{_{A}}y_{Gyr,A,t}−^{S}^{_{A}}b_{Gyro,A,t}

^{S}^{_{B}}ω_{B,t}=^{S}^{_{B}}y_{Gyr,B,t}−^{S}^{_{B}}b_{Gyro,B,t}

Where y_{acc} and y_{gyr} are defined as the signals from an accelerometer and gyroscope respectively (in m/s²) and (rad/s). The change in orientation between two time steps can be described with the quaternion:

$$Δ {q}_{A} = [ cos ( \frac{\vert T \cdot {}_{}^{{S}_{A}}{ω}{}_{A , t}^{} \vert}{2} ) \frac{T \cdot {}_{}^{{S}_{A}}{ω}{}_{A , t}^{}}{\vert T \cdot {}_{}^{{S}_{A}}{ω}{}_{A , t}^{} \vert} sin ( \frac{\vert T \cdot {}_{}^{{S}_{A}}{ω}{}_{A , t}^{} \vert}{2} ) ] \approx [ 1 \frac{T \cdot {}_{}^{{S}_{A}}{ω}{}_{A , t}^{}}{2} ]$$

$$Δ {q}_{B} = [ cos ( \frac{\vert T \cdot {}_{}^{{S}_{B}}{ω}{}_{B , t}^{} \vert}{2} ) \frac{T \cdot {}_{}^{{S}_{B}}{ω}{}_{B , t}^{}}{\vert T \cdot {}_{}^{{S}_{B}}{ω}{}_{B , t}^{} \vert} sin ( \frac{\vert T \cdot {}_{}^{{S}_{B}}{ω}{}_{B , t}^{} \vert}{2} ) ] \approx [ 1 \frac{T \cdot {}_{}^{{S}_{B}}{ω}{}_{B , t}^{}}{2} ]$$

Then calculating the next orientation is the quaternion multiplication:

q_{GS}_{_{A}}_{,t}=q_{GS}_{_{A}}_{,t-1}·Δq_{A}

q_{GS}_{_{B}}_{,t}=q_{GS}_{_{B}}_{,t-1}·Δq_{B}

The equations for predicting the new state vector are:

$${}^{G} Δ {p}_{t} = {}^{G} Δ {p}_{t - 1} + T \cdot {}^{G} Δ {v}_{t - 1} + \frac{1}{2} \cdot {T}^{2} ( {}_{}^{G}{a}{}_{{S}_{B} , t}^{} - {}_{}^{G}{a}{}_{{S}_{A} , t}^{} )$$

$${}^{G} Δ {v}_{t} = {}^{G} Δ {v}_{t - 1} + T \cdot ( {}_{}^{G}{a}{}_{{S}_{B} , t}^{} - {}_{}^{G}{a}{}_{{S}_{A} , t}^{} )$$

$${}_{}^{G}{a}{}_{{S}_{A} , Lowpass , t}^{} = {c}_{acc} \cdot {}_{}^{G}{a}{}_{{S}_{A} , Lowpass , t - 1}^{} + ( 1 - {c}_{acc} ) \cdot {}_{}^{G}{a}{}_{{S}_{A} , t}^{}$$

$${}_{}^{{S}_{A}}{θ}{}_{ɛ , A , t}^{} = Δ {R}_{{S}_{A}}^{T} \cdot {}_{}^{{S}_{A}}{θ}{}_{ɛ , t - 1}^{} + T \cdot {v}_{Gyro , t}$$

$${}_{}^{{S}_{A}}{b}{}_{ɛ , A , t}^{} = {}_{}^{{S}_{A}}{b}{}_{ɛ , A , t - 1}^{} + {w}_{GyroBiasNoise , t}$$

$${}_{}^{{S}_{B}}{θ}{}_{ɛ , B , t}^{} = Δ {R}_{{S}_{B}}^{T} \cdot {}_{}^{{S}_{B}}{θ}{}_{ɛ , t - 1}^{} + T \cdot {v}_{Gyro , t}$$

$${}_{}^{{S}_{B}}{b}{}_{ɛ , B , t}^{} = {}_{}^{{S}_{B}}{b}{}_{ɛ , B , t - 1}^{} + {w}_{GyroBiasNoise , t} \\ ( \begin{matrix}{}_{}^{G}{a}{}_{{S}_{A} , t}^{} = {}^{{GS}_{A}} \hat{R} [ {}_{}^{{S}_{A}}{y}{}_{Acc}^{} \times ] \cdot {θ}_{ɛ , t - ]} + {}^{{GS}_{A}} \hat{R} \cdot {}_{}^{{S}_{A}}{y}{}_{Acc}^{} + \\ {}^{G} g + {v}_{Acc , t} , ( similar for {}_{}^{G}{a}{}_{{S}_{B} , t}^{} )\end{matrix} )$$

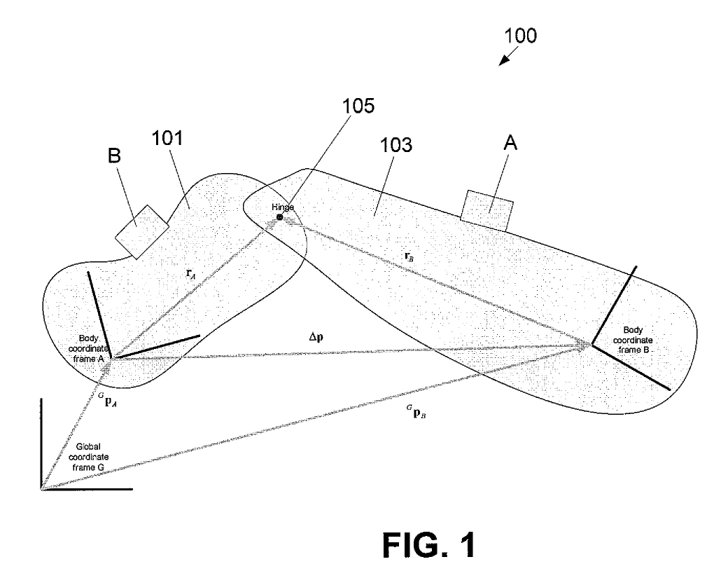

The manner in which the relative position is updated is illustrated in the segment diagram of FIG. 1. FIG. 1 shows an example of a body 100 consisting of 2 segments 101, 103 joined at a hinge 105. The position of sensor B on segment 101 is equal to the position of sensor A on segment 103 plus the relative distance between sensor A and sensor B, thus

$${}^{G} Δ {p}_{t} = {}_{}^{G}{p}{}_{B , t}^{} - {}_{}^{G}{p}{}_{A , t}^{}$$

$$with$$

$${}_{}^{G}{p}{}_{A , t}^{} = {}_{}^{G}{p}{}_{A , t - 1}^{} + {}_{}^{G}{v}{}_{A , t - 1}^{} \cdot T + \frac{1}{2} {}_{}^{G}{a}{}_{A , t - 1}^{} \cdot {T}^{2} ( similar for {}_{}^{G}{p}{}_{B , t}^{} )$$

These equations are implemented for updating the state vector. The covariance matrix is updated with the equation Q_{x,t+1}=A·Q_{x,t}·A′+Q_{w}, with A, the Jacobian matrix, given by:

$$A = [ \begin{matrix}{I}_{3} & T \cdot {I}_{3} & {O}_{3} & - \frac{1}{2} \cdot {T}^{2} \cdot ( {}^{{GS}_{A}} \hat{R} [ {}_{}^{{S}_{A}}{y}{}_{Acc}^{} \times ] ) & {O}_{3} & \frac{1}{2} \cdot {T}^{2} ( {}^{{GS}_{B}} \hat{R} [ {}_{}^{{S}_{B}}{y}{}_{Acc}^{} \times ] ) & {O}_{3} \\ {O}_{3} & {I}_{3} & {O}_{3} & - T \cdot ( {}^{{GS}_{A}} \hat{R} [ {}_{}^{{S}_{A}}{y}{}_{Acc}^{} \times ] ) & {O}_{3} & T \cdot ( {}^{{GS}_{B}} \hat{R} [ {}_{}^{{S}_{B}}{y}{}_{Acc}^{} \times ] ) & {O}_{3} \\ {O}_{3} & {O}_{3} & {c}_{acc} \cdot {I}_{3} & ( 1 - {c}_{acc} ) \cdot {}^{{GS}_{A}} \hat{R} [ {}_{}^{{S}_{A}}{y}{}_{Acc}^{} \times ] & {O}_{3} & {O}_{3} & {O}_{3} \\ {O}_{3} & {O}_{3} & {O}_{3} & Δ {R}_{{S}_{A}}^{T} & T \cdot {I}_{3} & {O}_{3} & {O}_{3} \\ {O}_{3} & {O}_{3} & {O}_{3} & {O}_{3} & {I}_{3} & {O}_{3} & {O}_{3} \\ {O}_{3} & {O}_{3} & {O}_{3} & {O}_{3} & {O}_{3} & Δ {R}_{{S}_{B}}^{T} & T \cdot {I}_{3} \\ {O}_{3} & {O}_{3} & {O}_{3} & {O}_{3} & {O}_{3} & {O}_{3} & {I}_{3}\end{matrix} ]$$

Similarly, the process noise covariance matrix is:

$${Q}_{w} = diag ( [ \begin{matrix}\frac{1}{4} \cdot {T}^{4} \cdot ( {Q}_{{vAcc\_S}_{A}} + {Q}_{{vAcc\_S}_{B}} ) \\ {T}^{2} \cdot ( {Q}_{{vAcc\_S}_{A}} + {Q}_{{vAcc\_S}_{B}} ) \\ {( 1 - {c}_{acc} )}^{2} \cdot {Q}_{{vAcc\_S}_{A}} \\ {T}^{2} \cdot {Q}_{{vGyr\_S}_{A}} \\ {Q}_{{vGyrBias\_S}_{A}} \\ {Q}_{{vGyr\_S}_{B}} \\ {Q}_{{vGyrBias\_S}_{B}}\end{matrix} ] )$$

It will be appreciated that the state and its covariance computed with dead reckoning can suffer from integration drift. This is optionally adjusted using the approximation that the average over time of the low passed acceleration in the global frame is zero, to obtain observability of inclination of the object segments:

$${a}_{Low , t} = [ \begin{matrix}\begin{matrix}0 \\ 0\end{matrix} \\ 0\end{matrix} ] + {w}_{Acc} , {W}_{Acc} • N ( 0 , {Q}_{wAcc} )$$

In an embodiment of the invention, this acceleration update is only performed for one of the units, e.g., sensor A.

Optionally, a magnetic field measurement update can be used for multiple sensors, such that when there is no joint acceleration (and the relative heading is not observable using the joint acceleration) the relative heading is not drifting and the rate gyroscopes biases remain observable.

The third measurement update uses the information that the two segments 101, 103 are connected by the joint 105. It follows from FIG. 1 that the distance between the joint 105 and sensor A, ^{S}r_{A} is equal to the relative position between sensor A and sensor B, Δp, plus the distance between the joint 105 and sensor B, ^{S}r_{B}. Thus Δp is equal to:

$${}^{G} Δ p = {}_{}^{G}{r}{}_{A}^{} - {}_{}^{G}{r}{}_{B}^{}$$

$${}^{G} Δ p = {}^{{GS}_{A}} \hat{R} ( I - [ {}_{}^{{S}_{A}}{θ}{}_{ɛ , A}^{} \times ] ) \cdot {}_{}^{{S}_{A}}{r}{}_{A}^{} - {}^{{GS}_{B}} \hat{R} ( I - [ {}_{}^{{S}_{B}}{θ}{}_{ɛ , B}^{} \times ] ) \cdot {}_{}^{{S}_{B}}{r}{}_{B}^{}$$

$${}^{G} Δ p = {}_{}^{G}{\hat{r}}{}_{A}^{} - {}^{{GS}_{A}} \hat{R} \cdot [ {}_{}^{{S}_{A}}{θ}{}_{ɛ , A}^{} \times ] \cdot {}_{}^{{S}_{A}}{r}{}_{A}^{} - {}_{}^{G}{\hat{r}}{}_{B}^{} + {}^{{GS}_{B}} \hat{R} [ {}_{}^{{S}_{B}}{θ}{}_{ɛ , B}^{} \times ] \cdot {}_{}^{{S}_{B}}{r}{}_{B}^{}$$

$${}^{G} Δ p = {}_{}^{G}{\hat{r}}{}_{A}^{} + {}^{{GS}_{A}} \hat{R} \cdot [ {}_{}^{{S}_{A}}{r}{}_{A}^{} \times ] \cdot {}_{}^{{S}_{A}}{θ}{}_{ɛ , A}^{} - {}_{}^{G}{\hat{r}}{}_{B}^{} - {}^{{GS}_{B}} \hat{R} [ {}_{}^{{S}_{B}}{r}{}_{B}^{} \times ] \cdot {}_{}^{{S}_{B}}{θ}{}_{ɛ , B}^{} \\ ⇓ \\ {}_{}^{G}{\hat{r}}{}_{A}^{} - {}_{}^{G}{\hat{r}}{}_{B}^{} = {}^{G} Δ p - {}^{{GS}_{A}} \hat{R} \cdot [ {}_{}^{{S}_{A}}{r}{}_{A}^{} \times ] \cdot {}_{}^{{S}_{A}}{θ}{}_{ɛ , A}^{} - {}^{{GS}_{B}} \hat{R} [ {}_{}^{{S}_{B}}{r}{}_{B}^{} \times ] \cdot {}_{}^{{S}_{B}}{θ}{}_{ɛ , B}^{}$$

The measurement update equations are than defined by:

y=^{GS}^{_{A}}\hat{R}·^{S}^{_{A}}r_{A}−^{GS}^{_{B}}\hat{R}·^{S}^{_{B}}r_{B}

C=[I₃ O₃ O₃−^{GS}^{_{A}}\hat{R}·[^{S}^{_{A}}r_{A}×] O₃ ^{GS}^{_{B}}\hat{R}·[^{S}^{_{B}}r_{B}×] O₃]

After the measurement updates, the estimates of the orientation errors, ^{S}^{_{A}}θ_{ε,A,t} and ^{S}^{_{B}}θ_{ε,B,t} are used to update the orientations q_{GS}_{_{A}}_{,t} and q_{GS}_{_{B}}_{,t}. The covariance matrix is updated accordingly and the orientation errors are set to zero. Additionally, the quaternions are normalized.

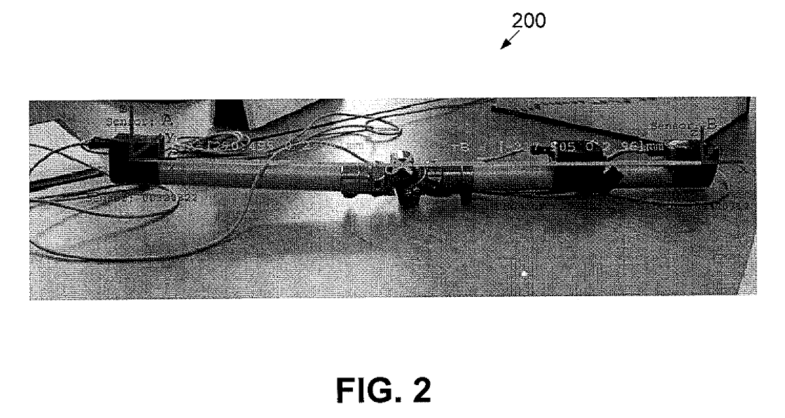

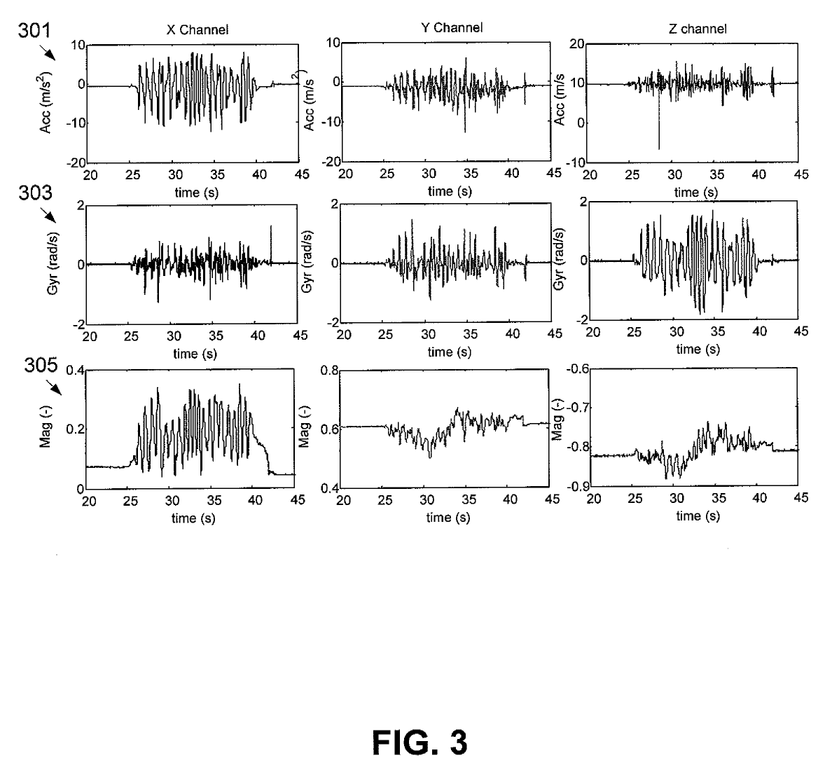

To test the algorithm, a measurement was preformed using a well-defined mechanical system, a prosthesis 200, as illustrated in FIG. 2. The prosthesis 200 in the measurement was initially lying still for the first 20 sec, then was translated in the x direction of the sensors, and then remained still again for 50 sec. The hinge (joint) angle was not changed during the experiment, and thus the relative position also was not changed. The calibrated data of sensor A and sensor B are shown in FIG. 3. The top row 301 of the graphs give the accelerometer signals, the middle row 303 of graphs the gyroscope signals and the bottom row 305 the magnetometer signals. The three columns of graphs give the signal along a different axis (x,y,z respectively).

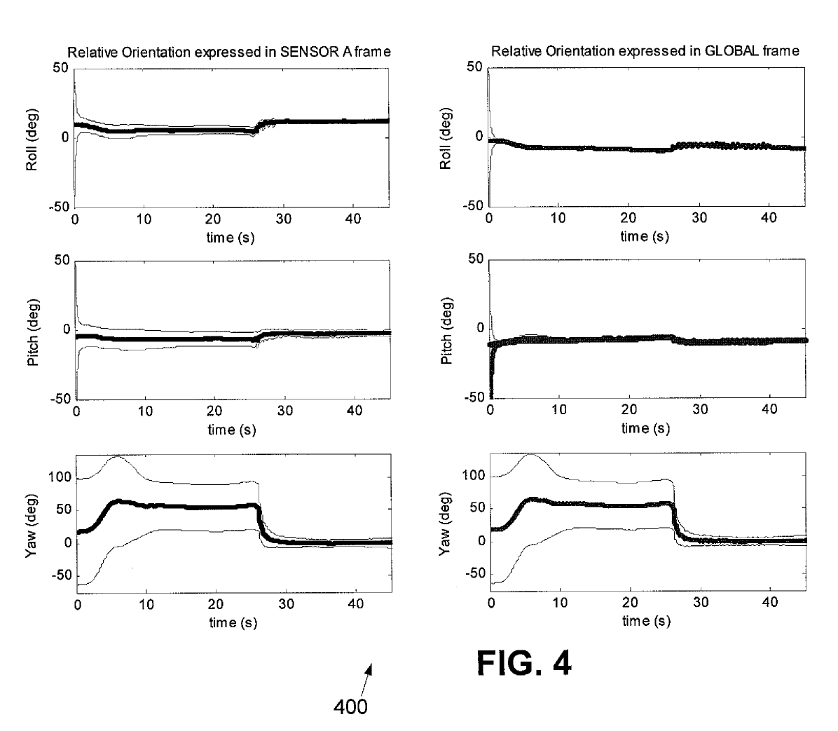

FIG. 4 illustrates a series of data plots 400 showing the orientation of sensor A in Euler angles (expressed in global frame) and the orientation of sensor B in Euler angles (expressed in global fame). From FIG. 4 it can be seen that the inclination of sensor A and the inclination of sensor B are observable immediately. In particular, the inclination of sensor A is directly observable due to the optional low pass acceleration update for sensor A, while the inclination of sensor B becomes observable due to the relative position update. The heading is not observable for both sensors when the sensors are lying still. This is because no information is given about the heading, e.g., no magnetometer measurement update was used.

From the foregoing, it will be appreciated that the relative heading becomes observable when the prosthesis is translated. The relative “heading” of the joint becomes observable when there are horizontal accelerations in the joint. In other words the relative heading is not observable when there is a perfect rotation around the joint centre or when there are only vertical accelerations (in the global frame), or when there is no movement or constant velocity (no acceleration) at all. To confirm this insight, several measurements were done where the prosthesis was rotated and translated.

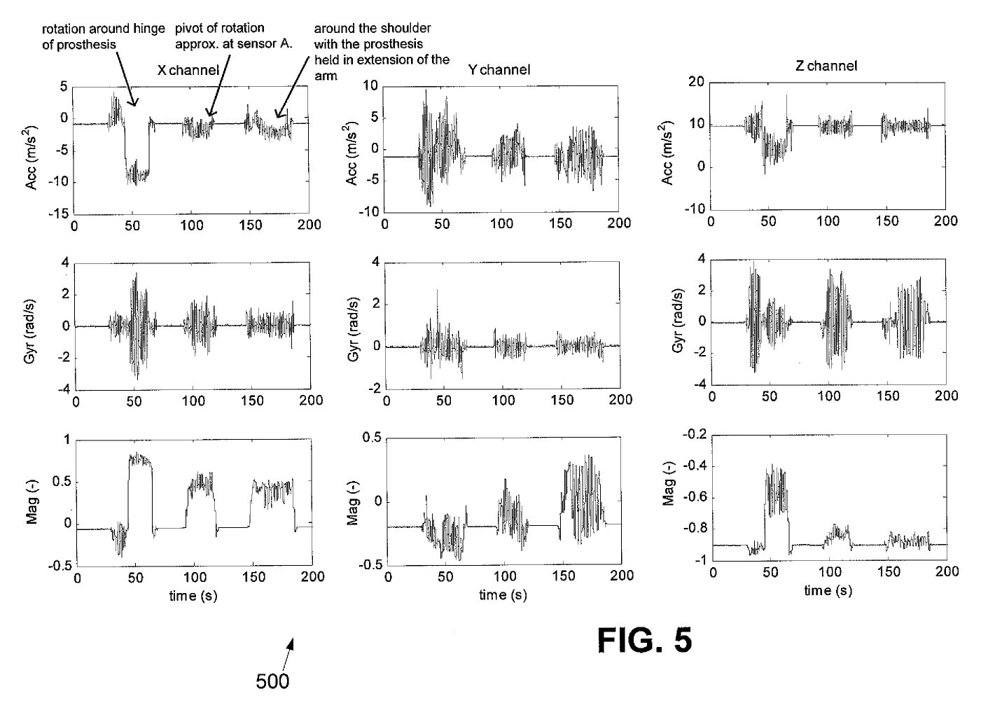

A measurement was performed where the prosthesis 200 was rotated around the hinge, around sensor A and around the shoulder with the prosthesis held in extension of the arm. In FIG. 5, the calibrated data 500 from this measurement as measured by a sensor A is shown, for the measurement where the prosthesis is rotated around the hinge, around sensor A and around the shoulder with the prosthesis held in extension of the arm.

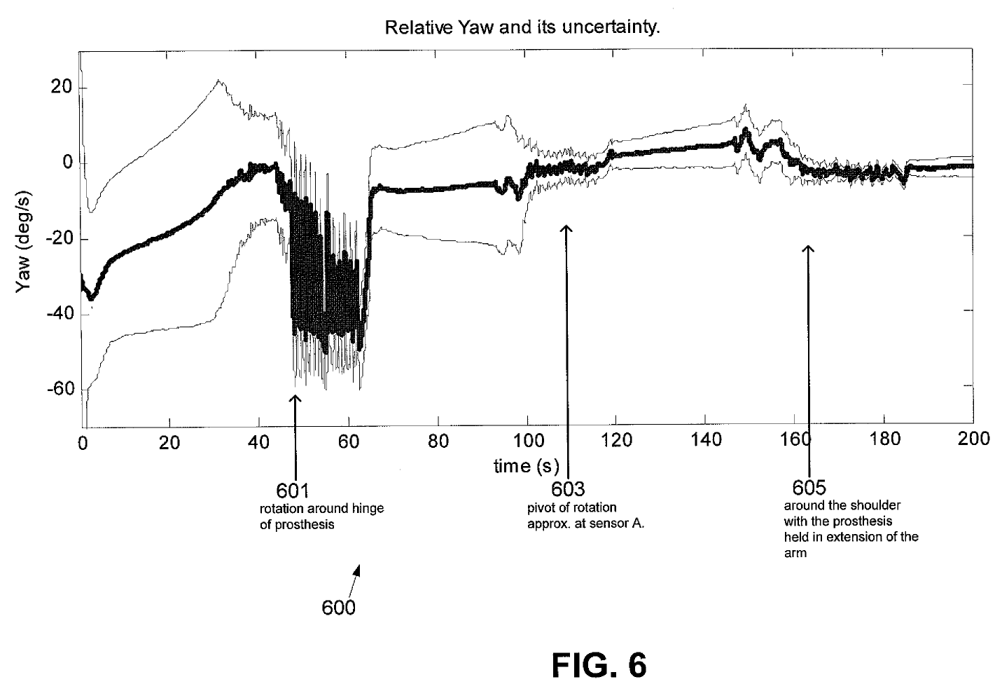

FIG. 6 shows the relative heading estimation 600, expressed in Global frame, for the three different types of rotations described in FIG. 5. The first section 601 gives the results of the rotation around the hinge, second section 603 plots of the rotation around sensor A and the third and last section 605 plots the results of the rotation around the shoulder. It is shown in FIG. 6 that it is difficult to converge to the correct relative heading when the prosthesis is rotated around the hinge, as can be observed from the relatively high yaw uncertainties.

Theoretically, the relative heading would not be observable, but due to the difficulty of a perfect rotation around the hinge centre there will be small net horizontal accelerations and therefore the relative heading can be roughly estimated. This illustrates the sensitivity of the method. For the rotation around sensor A and for the rotation around the shoulder, the relative heading estimation converges faster to the correct relative heading and the uncertainties are decreased as well.

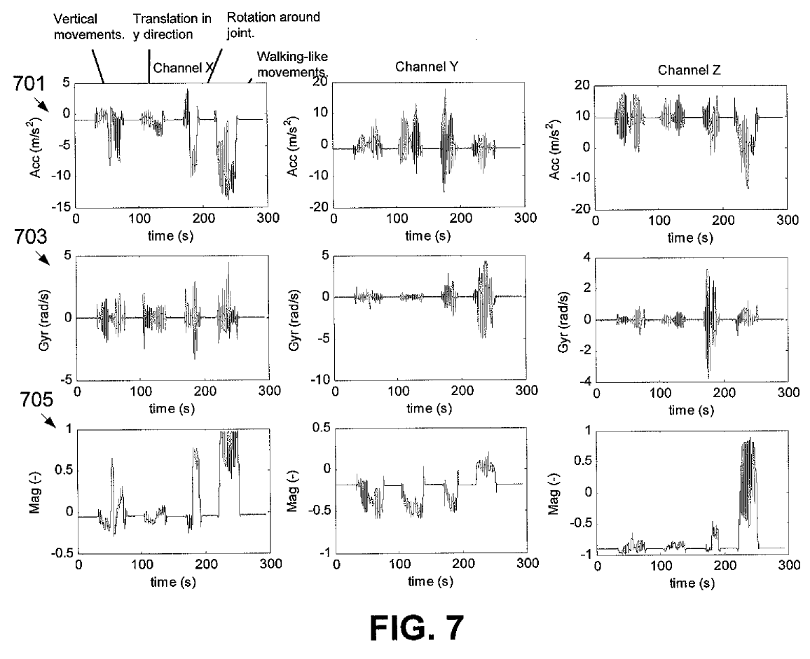

Subsequently, a measurement was done where the prosthesis 200 was translated along the x-, y- and z-axes. The calibrated data 700 from this measurement is shown in FIG. 7. The calibrated data measured by sensor A is shown. Each column gives data of a different axis (x,y,z); the top row 701 gives accelerometer signals, the middle row 703 gyroscope signals and the bottom row 705 magnetometer signals. Arrows indicate at what times the prosthesis is translated in the z-direction and in the y-direction, and rotated around the joint and rotated around sensor A with the joint free to move.

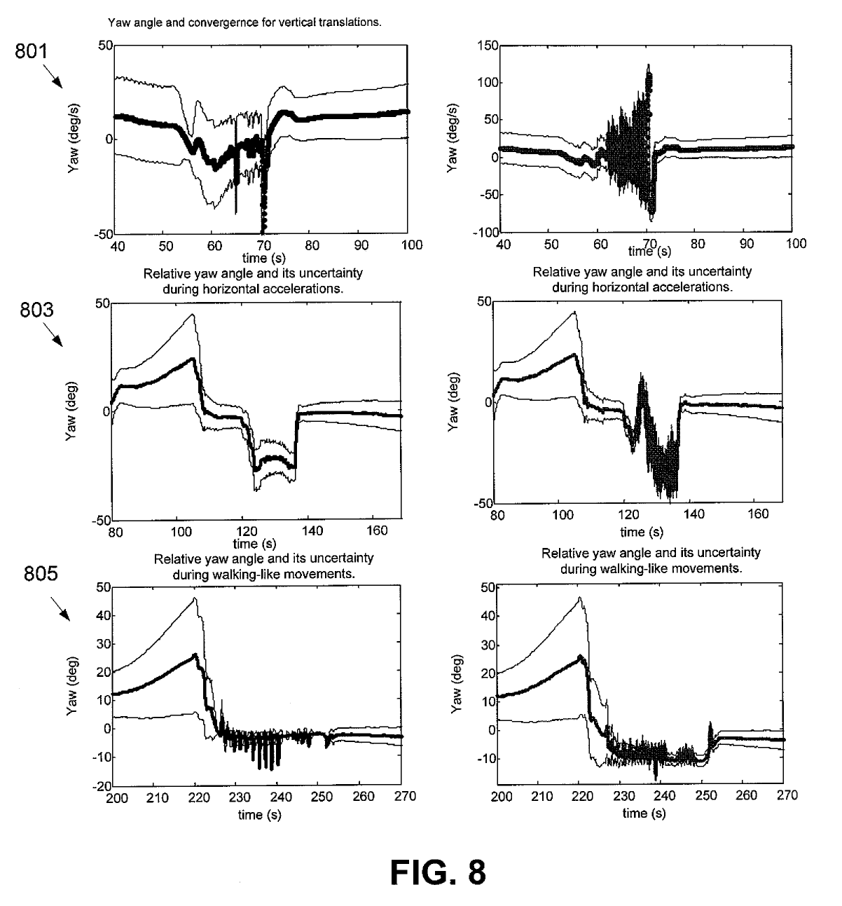

In FIG. 8, the relative heading estimation 800 is shown for several movements of the prosthesis using the trial described in FIG. 7. The top graphs 801 of FIG. 8 show the relative heading estimation (expressed in sensor frame A and expressed in Global frame) for translations in z-direction with a hinge angle of almost 180 degrees and with a hinge angle of about 90 degrees. The middle plots 803 are the results of translation in y-direction and the bottom plots 805 are the results of the rotation around sensor A with the joint free to move (simulated walking movement). These plots show that it is difficult to observe the relative heading for only translations in z-direction as can be seen from the relatively high uncertainty. For translations in the y-direction, the relative heading is observable as shown by the fast convergence when the translation starts and by the value of the minimal uncertainty.

For practical use, this concept as derived and demonstrated above can be extended to multiple segments, and indeed could be extended to an arbitrary number of joints and sensors. Also, it is not necessary in every embodiment to have a full IMU on each segment, or indeed a sensor at all on all segments. To demonstrate the practical application for a much used system of 3 segments connected by 2 joints, such as for example a leg or arm is derived below.

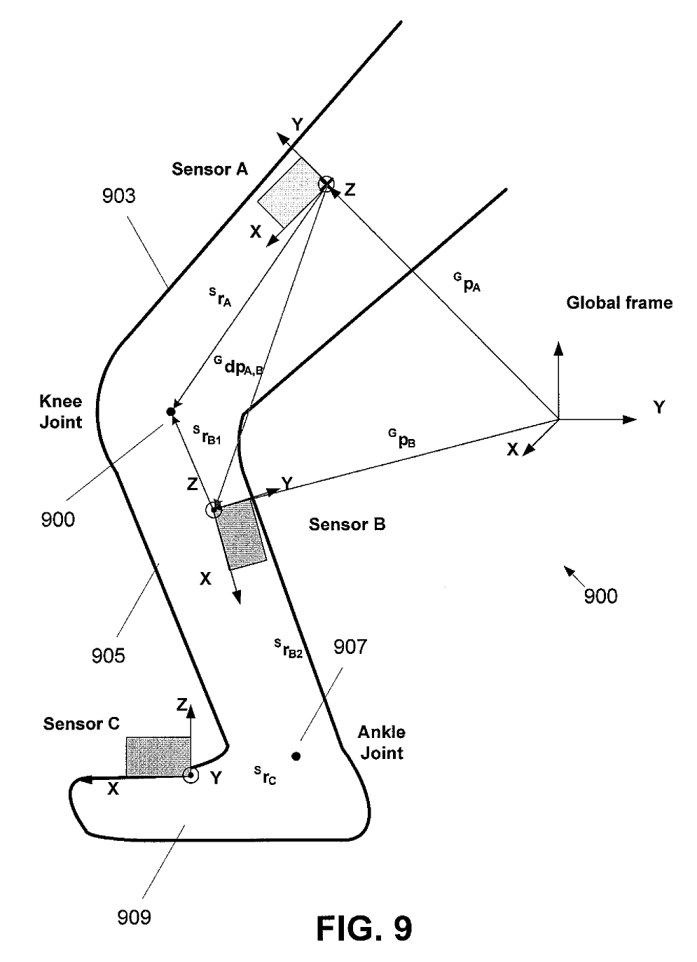

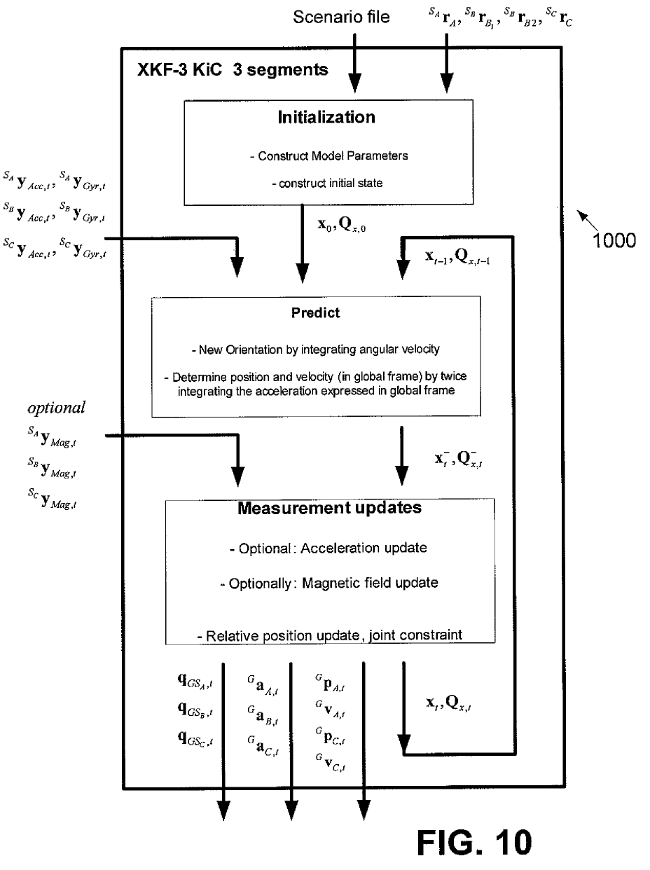

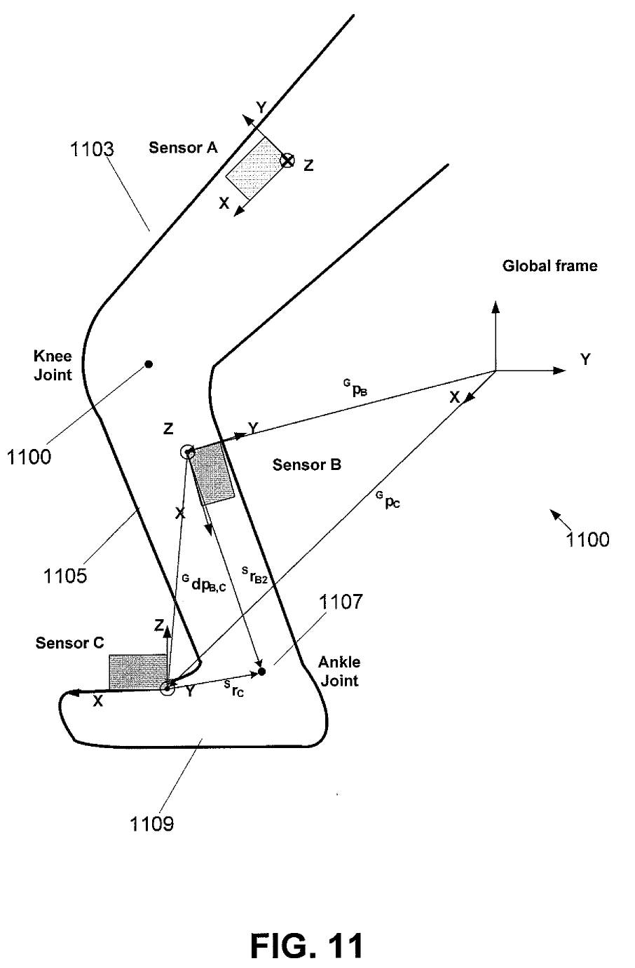

As example for demonstrating the KiC algorithm a leg 900 will be considered, e.g., the knee joint 901 connecting the upper leg (thigh) 903 with the lower leg (shank) 905 and the ankle joint 907 connecting the shank 905 with the foot 909 as shown in FIG. 9. The relations between the ankle joint 907, sensor B (attached to the shank 905) and sensor C (attached to the foot 909) are shown in FIG. 11, wherein like-ended numbers refer to like elements relative to FIG. 9. The “scenario file” 1000 illustrated in FIG. 10 shows a set of predetermined parameters for the algorithm.

The inputs
^{S}^{_{A}}U_{A}, ^{S}^{_{B}}U_{B} ^{S}^{_{C}}U_{C}, the calibrated data (acc, gyr, mag) of 3 IMUs expressed in the object coordinate frames;
^{S}r_{A}, ^{S}r_{B}_{₁}, ^{S}r_{B}_{₂}, ^{S}r_{C}, the joint positions expressed in the object coordinate frames; and
A scenario containing for example the initial settings of the algorithm and other parameters.

The outputs
positions of lump origins: ^{G}p_{A}, ^{G}p_{C};
velocity of lump origins: ^{G}v_{A}, ^{G}v_{C};
Orientation of the segments: q_{GS}_{_{A}}, q_{GS}_{_{B}}, q_{GS}_{_{C}}; and
Acceleration of the segments: ^{G}a_{A}, ^{G}a_{B}, ^{G}a_{C}.

The state vector consists of:
positions: ^{G}p_{A}, ^{G}p_{B}, ^{G}p_{C};
velocities: ^{G}v_{A}, ^{G}v_{B}, ^{G}v_{C};
a_lowpass: ^{G}a_{A,lowpass,t};
orientation errors: ^{S}^{_{A}}θ_{A}, ^{S}^{_{B}}θ_{B}, ^{S}^{_{C}}θ_{C};
gyro bias: ^{S}^{_{A}}b_{gyr,A}, ^{S}^{_{B}}b_{gyr,B}, ^{S}^{_{C}}b_{gyr,C}; and
magnetic field: ^{G}m_{A}, ^{G}m_{B}, ^{G}m_{C}.

In total, there are 16 state variables and 48 states. The equations for updating the state estimates are:

$$\begin{matrix}{}_{}^{G}{P}{}_{S , t}^{} = {}_{}^{G}{p}{}_{S , t - 1}^{} + T \cdot {}_{}^{G}{v}{}_{S , t - 1}^{} + \frac{1}{2} \cdot {T}^{2} \cdot {}_{}^{G}{a}{}_{S , t}^{} ( for all 3 sensors ) \\ {}_{}^{G}{v}{}_{S , t}^{} = {}_{}^{G}{v}{}_{S , t - 1}^{} + T \cdot {}_{}^{G}{a}{}_{S , t}^{} ( for all 3 sensors ) \\ {}_{}^{G}{a}{}_{A . Lowpass , t}^{} = {c}_{acc} \cdot {}_{}^{G}{a}{}_{A , Lowpass , t - 1}^{} + ( 1 - {c}_{acc} ) \cdot {}_{}^{G}{a}{}_{A , t}^{} \\ {}_{}^{S}{θ}{}_{ɛ , S , t}^{} = Δ {R}_{S}^{T} \cdot {}_{}^{S}{θ}{}_{ɛ , S , t - 1}^{} + T \cdot {v}_{Gyro , t} ( for all 3 sensors ) \\ {}_{}^{S}{b}{}_{ɛ , S , t}^{} = {}_{}^{S}{b}{}_{ɛ , S , t - 1}^{} + {w}_{GyroBiasNoise , t} ( for all 3 sensors ) \\ {}_{}^{G}{m}{}_{S , t}^{} = {c}_{mag} \cdot {}_{}^{G}{m}{}_{S , t - 1}^{} + ( 1 - {c}_{mag} ) \cdot {}_{}^{G}{m}{}_{S , mean}^{} ( optional ) \\ ( \begin{matrix}{}_{}^{G}{a}{}_{S , t}^{} = {}^{GS} \hat{R} [ {}_{}^{S}{y}{}_{Acc}^{} \times ] \cdot {θ}_{ɛ , S , t - 1} + {}^{GS} \hat{R} \cdot {}_{}^{S}{y}{}_{Acc}^{} + {}^{G} g + {v}_{Acc , t} \\ {}_{}^{G}{m}{}_{S , mean}^{} = {[ \sqrt{{}_{}^{G}{m}{}_{S , x , Lowpass}^{2} + {}_{}^{G}{m}{}_{S , y , Lowpass}^{2}} 0 {}_{}^{G}{m}{}_{S , z , t - 1}^{} ]}^{′}\end{matrix} ) \\ {}_{}^{G}{p}{}_{A , t}^{} = {}_{}^{G}{p}{}_{A , t - 1}^{} + T \cdot {}_{}^{G}{v}{}_{A , t - 1}^{} + \frac{1}{2} \cdot {T}^{2} {}_{}^{G}{a}{}_{S , t - 1}^{} ( for all 3 sensors )\end{matrix}$$

### Using the Relative Position Update

If inclination (roll/pitch) of 1 sensor is known, the inclination of the other sensor become observable;
Sensors measure the acceleration of the joint. If the acceleration of the joint is in the horizontal plane, the relative heading is observable;
If the heading of 1 sensor is known, the heading of the other sensors become observable.

There are several ways for writing down the relations for three segments connected by two joints.

The notion in the picture:
^{S}r_{A} is the joint position (connected to the segment to which sensor A is attached) expressed in the coordinate frame of sensor A (the vector from the origin of the sensor A frame to the joint position).
^{G}p_{A} is the origin of sensor A expressed in the global frame.
^{G}Δp_{A,B} is the vector from the origin of sensor A to the origin of sensor B or the position of sensor B expressed in the coordinate frame of sensor A.

Measurement update 1:

^{G}p_{B}−^{G}p_{A}=^{G}r_{A}−^{G}r_{B}_{₁}

When the state vector is known, the C matrix etc can be constructed via the equation below:

$${}_{}^{G}{p}{}_{B}^{} - {}_{}^{G}{p}{}_{A}^{} = {}^{GS} \hat{R} ( I - [ {}_{}^{{S}_{A}}{θ}{}_{ɛ , A}^{} \times ] ) \cdot {}_{}^{{S}_{A}}{r}{}_{A}^{} - {}^{{GS}_{B}} \hat{R} ( I - [ {}_{}^{{S}_{B}}{θ}{}_{ɛ , B}^{} \times ] ) \cdot {}_{}^{{S}_{B}}{r}{}_{{B}_{1}}^{}$$

$${}_{}^{G}{p}{}_{B}^{} - {}_{}^{G}{p}{}_{A}^{} = {}_{}^{G}{\hat{r}}{}_{A}^{} - {}_{}^{G}{\hat{r}}{}_{{B}_{1}}^{} - {}^{{GS}_{A}} \hat{R} \cdot [ {}_{}^{{S}_{A}}{θ}{}_{ɛ , A}^{} \times ] \cdot {}_{}^{{S}_{A}}{r}{}_{A}^{} + {}^{{GS}_{B}} \hat{R} \cdot [ {}_{}^{{S}_{B}}{θ}{}_{ɛ , B}^{} \times ] \cdot {}_{}^{{S}_{B}}{r}{}_{{B}_{1}}^{}$$

$${}_{}^{G}{p}{}_{B}^{} - {}_{}^{G}{p}{}_{A}^{} = {}_{}^{G}{\hat{r}}{}_{A}^{} - {}_{}^{G}{\hat{r}}{}_{{B}_{1}}^{} + {}^{{GS}_{A}} \hat{R} \cdot [ {}_{}^{{S}_{A}}{r}{}_{A}^{} \times ] \cdot {}_{}^{{S}_{A}}{θ}{}_{ɛ , A}^{} - {}^{{GS}_{B}} \hat{R} \cdot [ {}_{}^{{S}_{B}}{r}{}_{{B}_{1}}^{} \times ] \cdot {}_{}^{{S}_{B}}{θ}{}_{ɛ , B}^{} \\ ⇓ \\ {}_{}^{G}{\hat{r}}{}_{A}^{} - {}_{}^{G}{\hat{r}}{}_{{B}_{1}}^{} = {}^{G} {P}_{B} - {}_{}^{G}{p}{}_{A}^{} - {}^{{GS}_{A}} \hat{R} \cdot [ {}_{}^{{S}_{A}}{r}{}_{A}^{} \times ] \cdot {}_{}^{{S}_{A}}{θ}{}_{ɛ , A}^{} + {}^{{GS}_{B}} \hat{R} \cdot [ {}_{}^{{S}_{B}}{r}{}_{{B}_{1}}^{} \times ] \cdot {}_{}^{{S}_{B}}{θ}{}_{ɛ , B}^{}$$

The state variables concerning this update are:

^{G}p_{A}, ^{G}p_{B}, ^{G}v_{A}, ^{G}v_{B}, ^{S}^{_{A}}r_{A}, ^{S}^{_{B}}r_{B}_{₁}, ^{S}^{_{A}}θ_{A}, ^{S}^{_{B}}θ_{B}

Measurement update 2:

^{G}p_{C}−^{G}p_{B}=^{G}r_{B}_{₂}−^{G}r_{C}:

^{G}\hat{r}_{B2}−^{G}\hat{r}_{C}=^{G}p_{C}−^{G}p_{B}−^{GS}^{_{B}}\hat{R}·[^{S}^{_{B}}r_{B}_{₂}×]·^{S}^{_{B}}θ_{ε,B}+^{GS}^{_{C}}\hat{R}·[^{S}^{_{C}}r_{C}×]·^{S}^{_{C}}θ_{ε,C}

When the state vector is known, the C matrix etc can be constructed given the equation above.

The state variables concerning this update are:

^{G}p_{B}, ^{G}p_{C}, ^{G}v_{B}, ^{G}v_{C}, ^{S}^{_{B}}r_{B2}, ^{S}^{_{C}}r_{C}, ^{S}^{_{B}}θ_{B}, ^{S}^{_{C}}θ_{C}

The measurement update assuming, that the average acceleration in the global frame over some time is zero, optionally need only to be applied done for one sensor, for example sensor A, the sensor mounted to the upper leg.

The joint is defined in a rather general manner: If two segments are said to share a joint, there exist a point on each of the two segments that have zero average displacement with respect to each other, over a pre-determined period of time. The location of this point is the joint position. The location of this point may change as a function of time or joint angle. Put in a different way, a joint is described as a ball and socket containing some positional laxity. As the segments on each side of the joint are assumed to be rigid, the position of this point is usually fixed and can be expressed with respect to segment (object) coordinates.

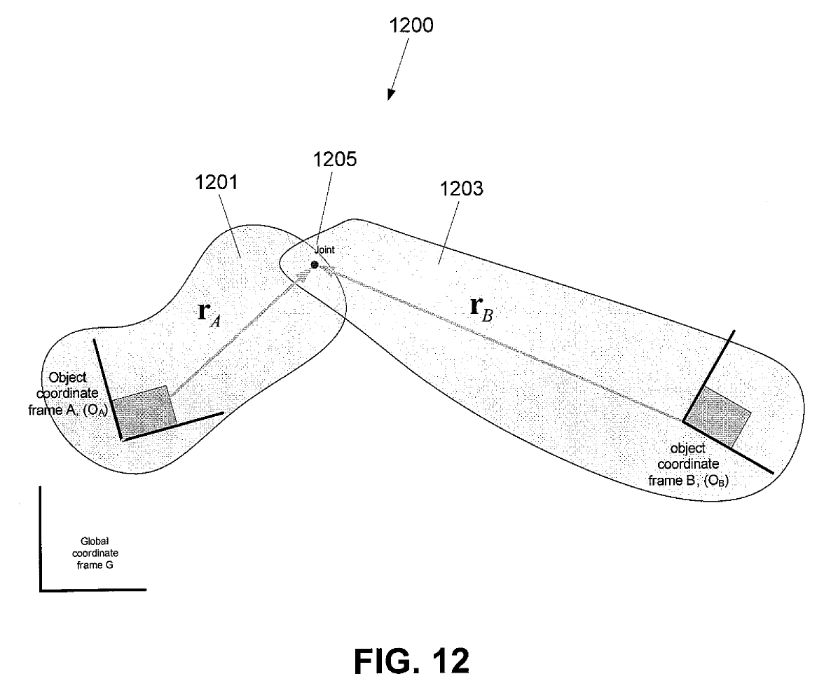

This can be seen in the example 1200 of FIG. 12, wherein two rigid segments A 1201 and B 1203 are connected by a joint 1205. An IMU is rigidly attached to each segment. In this figure, the object coordinate frame is the same as the sensor coordinate frame, the default case. r_{A} is the joint position expressed in object frame Q and r_{B} is the joint position expressed in object frame B.

Using the relation of the Kinematic Coupling, the algorithm is able to supply the relative orientation between the two segments without using any assumptions on the local magnetic field during movements:

From the assumption that two segments are connected by a joint it follows that the acceleration of the joint is equal to the acceleration measured by the IMU's attached to the segments expressed in the joint position and expressed in the global coordinate frame. Or in other words, both IMUs should measure the same acceleration in the joint. This is demonstrated above.

If, for example, the orientation of the IMU attached to segment A is known, then the acceleration measured by this IMU can be expressed in the global coordinate frame and translated to the joint. Because the acceleration in the joint measured by the IMU attached to segment B must be equal to the acceleration measured by the IMU attached to segment A, the relative orientation, including rotation around the vertical, of the IMU attached to segment B is known, without using any information of the magnetometers. This method assumes that the location of the joint with respect to the IMUs (rA and rB) is known.

There is one important exception to the above: the relative orientation between the two segments can only be determined if the joint occasionally experiences some horizontal acceleration, e.g., during walking. The duration of such periods depends on the movement, the amount of correction needed due to rate gyroscope integration drift, the uncertainties of assumptions being made and settling time. For the case of the knee joint, a few steps of walking every 30 seconds would be sufficient for typical low-grade automotive rate gyros. In case the knee is not moving for much more than half a minute, the local relative heading could still be determined using the earth magnetic field, or optionally only used to limit any drift and make the rate gyro bias observable.

The accuracy of the joint position estimate with respect to the positions of the sensors on the segment should be known a priori, but, depending on the accuracy needed, does not need to be determined better than within 2-3 cm.

The inputs for the KiC Algorithm are:
The calibrated data of two IMUs expressed in the object coordinate frames,
The joint position expressed in both object coordinate frames,
A scenario containing for example the initial settings of the algorithm.

The KiC algorithm assumes the distances between the joint and the origin of the IMUs attached to the segments to be known. Therefore the vector expressing the joint position in the object coordinate frame of segment A, OA, and the vector expressing the joint position in the object coordinate frame of segment B, OB, need to be given as input. These two vectors have to be set by the user. They can be obtained e.g., by measuring the joint position using a measuring tape.

A “scenario” controls the settings, e.g., the optional use of magnetometers, tuning parameters and initial settings used in the KiC algorithm. It specifies the characteristics of the movement and also parameters describing the uncertainties of assumptions being made.

Additionally, it can be shown using the above methods than instead of assuming the distance between the sensor A and sensor B and the joint to be known a priori, it can be left to the algorithm to estimate these distances. The disadvantage for this approach is that the distances in the state vector only become accurately observable when the system is excited enough. This may not be the case for a typical application and it will cause the algorithm to converge very slowly. Additionally, often the mounting location of the sensors with respect to the joint can be easily known, at least roughly. A huge advantage by letting the system automatically estimate the distances while using the system is that it can be very hard or impossible to actually measure the joint location accurately. This is also discussed above.

Furthermore, additional constraints can be added to the joint properties, e.g., a hinge with only 1 or 2 degrees of freedom, or other (mechanical) models can be used. Effectively this reduces the degrees of freedom of the joint and adds observability to the relative orientation estimates and/or the estimate of the distance between the IMUs end the joints. This can be advantageous in systems, such as such as prostheses, with well defined joints. However, it should be used with care for less defined systems, such as human joints, since an erroneous assumption will influence the accuracy of the system negatively.

In addition, the joint acceleration measurements can be further improved by combining the above described methods with other systems that can measure position, velocity and/or acceleration. For example UWB positioning systems or camera based systems can be used as input for a more accurate position/velocity/acceleration measurement.

It will be appreciated that the exact location of the accelerometer cluster inside the IMU is not critical, but the size of the accelerometer cluster inside the IMU should preferably be compensated for. It will be further appreciated that the disclosed principles have application far beyond measuring human motion. Indeed, the disclosed principles can be applied in any system that consists of one or more bodies comprising different segments connected by joints. Example environments for application of the disclosed principles include robots, sailing boats, cranes, trains, etc.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Certain examples of the invention are described herein, including the best mode known to the inventors for carrying out the invention. Variations of those examples will be apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of measuring the motion of an object composed of multiple segments connected by joints via the estimation of the 3D orientation of the object segments relative to one another, without dependence on the Earth magnetic field as a reference for heading, the method comprising:
applying a plurality of inertial sensor units to respective ones of the multiple segments;
subjecting the joint to an acceleration;
calculating the relative orientation of each segment with respect to each other based on data from the sensor units; and
using the orientations of the segments to form an estimation of the 3D orientation of the object segments relative to one another without using the local magnetic field as a reference for heading.

2. The method of measuring the motion of an object according to claim 1, wherein calculating the relative orientation of each segment further comprises comparing the measured accelerations from a first inertial sensor and a second inertial sensor at the location of the joint.

3. The method of measuring the motion of an object according to claim 1, further comprising calculating the distance between each sensor and each adjacent joint based on data from the sensors.

4. The method of measuring the motion of an object according to claim 1, wherein using the orientations of the segments without using the local magnetic field as a reference for heading comprises calculating position and orientation of the object.

5. The method of measuring the motion of an object according to claim 1, wherein the object is a human body.

6. The method of measuring the motion of an object according to claim 1, further comprising providing the estimation of 3D orientation to one of a motion capture system, Virtual Reality system and an Augmented Reality system.

7. The method of measuring the motion of an object according to claim 1, wherein the object is a robotic device.

8. A computer-readable medium having thereon computer-executable instructions for measuring the motion of an object composed of multiple segments connected by joints via the estimation of the 3D orientation of the object segments relative to one another, without dependence on the Earth magnetic field as a reference for heading, the object having a plurality of inertial sensor units affixed thereto upon respective ones of the multiple segments, the computer-executable instructions comprising:
instructions for receiving data from one or more of the inertial sensor units indicating that that one or more joints have been subjected to acceleration;
instructions for calculating the relative orientation of each segment with respect to each other based on the received data from the sensor units; and
instructions for using the orientations of the segments to form an estimation of the 3D orientation of the object segments relative to one another without using the local magnetic field as a reference for heading.

9. The computer-readable medium according to claim 8, wherein the instructions for calculating the relative orientation of each segment further comprise instructions for comparing the measured accelerations from a first inertial sensor and a second inertial sensor at the location of the joint.

10. The computer-readable medium according to claim 8, further comprising instructions for calculating the distance between each sensor and each adjacent joint based on data from the sensors.

11. The computer-readable medium according to claim 8, wherein the instructions for using the orientations of the segments without using the local magnetic field as a reference for heading comprise instructions for calculating position and orientation of the object.

12. The computer-readable medium according to claim 8, wherein the object is a human body.

13. The computer-readable medium according to claim 8, further comprising instructions for providing the estimation of 3D orientation to one of a motion capture system, Virtual Reality system and an Augmented Reality system.

14. The computer-readable medium according to claim 8, wherein the object is a robotic device.
